(19) United States
(12) Patent Application Publication
(10) Pub. No.: US 2025/0026704 A1
(43) Pub. Date: Jan. 23, 2025

(54) PROCESS FOR HYDROFORMYLATION OF DIISOBUTENE AND A C4 TO C7 OLEFIN

(71) Applicant: Evonik Oxeno GmbH & Co. KG, Marl (DE)

(72) Inventors: Peter KUCMIERCZYK, Herne (DE); Maren TERMÜHLEN, Dortmund (DE); Annika REINHARDT, Marl (DE); Armin Matthias RIX, Marl (DE); Robert FRANKE, Marl (DE); Dirk FRIDAG, Haltern am See (DE); Anna Chiara SALE, Düsseldorf (DE); Ana MARKOVIC, Haltern am See (DE)

(73) Assignee: Evonik Oxeno GmbH & Co. KG, Marl (DE)

(21) Appl. No.: 18/776,372

(22) Filed: Jul. 18, 2024

(30) Foreign Application Priority Data
Jul. 20, 2023 (EP) 23186740.9

(51) Int. Cl. C07C 45/50; B01D 3/14; B01J 31/18; B01J 31/24; C07C 45/78; C07C 45/82

(52) U.S. Cl. CPC C07C 45/505; B01D 3/14; B01J 31/185; B01J 31/24; C07C 45/786; C07C 45/82; B01J 2231/321; B01J 2531/004; B01J 2531/822

(57) ABSTRACT

The invention provides a process for hydroformylation of diisobutene and a C4 to C7 olefin in a common reaction zone. The hydroformylation is carried out with synthesis gas in the presence of a homogeneous catalyst system that comprises at least Co or Rh and optionally a phosphorus-containing ligand.

The present invention relates to a process for hydroformylation of diisobutene and a C4 to C7 olefin in a common reaction zone. The hydroformylation is carried out with synthesis gas in the presence of a homogeneous catalyst system that comprises at least Co or Rh and optionally a phosphorus-containing ligand.

Diisobutene is an industrially relevant product obtained by dimerization of isobutene. Diisobutene consists of the isomers 2,4,4-trimethylpent-1-ene (hereinbelow also: TMP1) and 2,4,4-trimethylpent-2-ene (hereinbelow also: TMP2) with a mass distribution of TMP1:TMP2 in the range of about 78:22 to 81:19 (equilibrium distribution). Industrial mixtures comprising C4 olefins are light petroleum fractions from refineries, C4 fractions from FC crackers or steam crackers, mixtures from Fischer-Tropsch syntheses, mixtures from dehydrogenation of butanes, and mixtures resulting from metathesis or other industrial processes. C5 olefins, i.e. pentenes, are present in light petroleum fractions from refineries or crackers. The higher olefins are obtainable in particular by oligomerization reactions. Both diisobutene and C4 to C7 olefins may be converted by the hydroformylation into valuable products such as the esters formed in the hydroformylation.

The problem with such processes is that dedicated production plants must be available or constructed and that these plants must be operated with the costs that this entails. Since the markets for petrochemical products are in some cases rather variable, it is hardly possible to operate dedicated production plants for each of the recited olefins economically. A further disadvantage is that resource-efficient operation of several production plants is hardly possible because all plants require maintenance. This entails not only economic and personnel costs but also necessitates certain amounts of energy such as electricity or heat transfer medium.

It is accordingly an object of the present invention to provide a process which does not exhibit the aforementioned problems. It should especially be possible to convert both diisobutene and C4 to C7 olefins into valuable products via hydroformylation in a more resource-efficient manner.

The underlying object was achieved by the process described in Claim 1. Preferred embodiments are specified in the dependent claims.

According to the invention the process for hydroformylation of diisobutene and a C4 to C7 olefin comprises at least the following steps:
a. providing a diisobutene stream containing 2,4,4-trimethylpent-2-ene and 2,4,4-trimethylpent-1-ene and providing an olefin stream containing the C4 to C7 olefin;
b. hydroformylation of diisobutene and the C4 to C7 olefin with synthesis gas in the presence of a homogeneous catalyst system comprising at least Co or Rh and optionally a phosphorus-containing ligand in a reaction zone to obtain a, preferably liquid product mixture comprising at least the aldehydes 3,5,5-trimethylhexanal and a C5 to C8 aldehyde formed by the hydroformylation, the homogeneous catalyst system and unreacted olefins, i.e. unreacted diisobutene and unreacted C4 to C7 olefins;
c. removing the homogeneous catalyst system from the preferably liquid product mixture to obtain a crude product mixture comprising at least the aldehydes 3,5,5-trimethylhexanal and a C5 to C8 aldehyde formed by the hydroformylation and the unreacted olefins; and
d. distillative processing of the crude product mixture in at least one distillation column to remove the unreacted olefins to obtain an aldehyde mixture containing the aldehydes 3,5,5-trimethylhexanal and a C5 to C8 aldehyde formed, wherein preferably the unreacted olefins are removed and recycled to the hydroformylation in step b.

The process according to the invention therefore relates to the simultaneous reaction of diisobutene and C4 to C7 olefins in a single common reaction zone. Such a procedure has numerous advantages.

The process described makes it possible to react flexibly to markets, in particular small production amounts. In addition, only one production plant is necessary and said plant can also be operated more efficiently and thus in a more resource-saving manner even when market requirements fluctuate. The specific boiling order also makes it possible to separate the obtained products of the respectively employed olefins while the reactants may be returned to the reaction directly or after additional processing.

The diisobutene stream provided in step a comprises 2,4,4-trimethylpent-2-ene and 2,4,4-trimethylpent-1-ene. In a preferred embodiment, the proportion of 2,4,4-trimethylpent-1-ene in the diisobutene stream is at least 60 mol %, preferably at least 70 mol %, based on the total diisobutene stream. Such streams may be diisobutene streams produced by dimerization from isobutene or isobutene-containing hydrocarbon mixtures, for example according to the process disclosed in EP 1 360 160 B1. In addition, the diisobutene streams to be employed here may be obtained as unreacted residual streams in carbonylation processes, for example in alkoxycarbonylation or in hydroformylation.

Step a comprises providing not only the diisobutene stream but also an olefin stream which contains the C4 to C7 olefin employed in the process according to the invention. In a preferred embodiment the present invention employs an olefin stream comprising C4 olefins, particularly preferably a C4 olefin stream. Corresponding streams are known to those skilled in the art and available on a large industrial scale.

Olefin streams comprising C4 olefins include for example light petroleum fractions from refineries, C4 fractions from FC crackers or steam crackers, mixtures from Fischer-Tropsch syntheses, mixtures from dehydrogenation of butanes or streams resulting from metathesis or from other industrial processes. For example, C4 olefin streams suitable for the process according to the invention are obtainable from the C4 fraction of a steam cracker. C5 olefins, i.e. pentenes, are present in light petroleum fractions from refineries or crackers. C6 olefins are obtainable for example by dimerization of propene. C7 olefins are obtainable for example by dimerization of propylene and butene.

The streams provided in step a, i.e. the diisobutene stream and the C4 to C7 olefin stream, are passed to the hydroformylation in step b. The streams may be individually and separately sent to the hydroformylation in step b or mixed beforehand. The diisobutene stream and the C4 to C7 olefin stream are preferably mixed prior to the hydroformylation in step b. A particularly preferred embodiment of the present invention in fact provides that the diisobutene stream, the C4 to C7 olefin stream and the homogeneous catalyst system are mixed, in particular in a suitable mixing vessel, prior to the hydroformylation in step b. If there is a recycle stream to the reaction, i.e. by recycling the catalyst system for example, this recycle stream can likewise be passed to the mixing vessel.

Step b comprises reacting the diisobutenes, i.e. 2,4,4-trimethylpent-2-ene and 2,4,4-trimethylpent-1-ene, with synthesis gas (mixture of carbon monoxide (CO) and hydrogen (H₂)) to afford an aldehyde. The number of carbon atoms in the aldehyde thus increases, compared to the diisobutene used, by 1 carbon atom. The diisobutenes (8 carbon atoms) accordingly give rise to an aldehyde having 9 carbon atoms, namely 3,5,5-trimethylhexanal. Accordingly the hydroformylation according to the invention converts the C4 to C7 olefin into a C5 to C8 aldehyde.

The synthesis gas for the process according to the invention may be employed in different mixing ratios of carbon monoxide and hydrogen. The molar ratio between synthesis gas and the employed hydrocarbon stream containing the olefins to be hydroformylated should be between 6:1 and 1:1, preferably between 3:1 and 1:1, particularly preferably between 2:1 and 1:1.

The hydroformylation may optionally be performed in the presence of an additional solvent known to those skilled in the art, though it is preferable when no additional solvent is used and the employed olefin functions as solvent in the hydroformylation.

The homogeneous catalyst system employable in the hydroformylation contains Co or Rh, preferably Rh, and optionally a phosphorus-containing ligand. Corresponding catalyst systems are familiar to those skilled in the art. The use of a phosphorus-containing ligand is preferred.

In a particularly preferred embodiment the homogeneous catalyst system comprises or consists of Rh and a phosphorus-containing ligand. Suitable ligands for the catalyst systems according to the invention are known to those skilled in the art. The phosphorus-containing ligand for the catalyst system according to the invention is preferably a phosphine (for example TPP (triphenylphosphine), a monophosphite (for example Alkanox 240 (tris(2,4-di-tert-butylphenyl)phosphite) or a bisphosphite (for example biphephos). It is also possible to employ mixtures of ligands.

The temperature during the homogeneously catalyzed hydroformylation is preferably in the range from 80° C. to 250° C., further preferably in the range from 90° C. to 225° C. and particularly preferably in the range from 100° C. to 210° C. The pressure during the homogeneously catalyzed hydroformylation is preferably in the range from 100 to 350 bar, further preferably in the range from 175 to 325 bar and particularly preferably in the range from 200 to 300 bar.

The pressure during the hydroformylation typically corresponds to the total gas pressure. In the context of the present invention the total gas pressure is to be understood as meaning the sum of the prevailing pressures of all present gaseous substances, i.e. the pressure of the (total) gas phase. In the present process this especially corresponds to the sum of the partial pressures of CO and H₂, i.e. the total gas pressure is then the synthesis gas pressure.

Homogeneously catalyzed hydroformylations may be operated as liquid output processes (“liquid recycle”) or as gas output processes (“gas recycle”). Both process variants are known to those skilled in the art and described in many textbooks. A specific selection of such a process is unnecessary in the context of the present invention because the process may in principle be performed in both ways. What is in any case important in homogeneous catalysis is the separation of the catalyst system from the reaction output. In the case of a liquid output this is possible for example via flash processes or membrane separation. In the case of a gaseous output for example by condensation and/or scrubbing. This too is known to those skilled in the art and does not require comprehensive explanation. The further workup of the reaction output, in particular the separation of the reaction product, is likewise familiar to those skilled in the art and may be carried out for example by means of a thermal separation process such as distillation. Thermal separation/thermal separation processes in the context of the present invention is to be understood as meaning separation processes where separation is effected by means of the boiling point.

The hydroformylation in step b takes place in a suitable reaction zone. The reaction zone for the reaction comprises at least one reactor, but may also consist of two or more reactors arranged in parallel or in series. The at least one reactor may in particular be selected from the group consisting of a stirred-tank reactor, a loop reactor, a jet-loop reactor, a bubble-column reactor or combinations thereof. If more than one reactor is used, the reactors may be identical or different.

The hydroformylation in step b described affords a preferably liquid product mixture that comprises at least the aldehydes 3,5,5-trimethylhexanal and the C5 to C8 aldehyde formed by the hydroformylation, the homogeneous catalyst system and the unreacted olefins, i.e. diisobutenes and C4 to C7 olefins.

The preferably liquid product mixture thus obtained is supplied to the following step c in order to remove the homogeneous catalyst system from the product mixture. The supplying of the product mixture may already be preceded by removal of low-boiling components, for example low-boiling by-products, for example by thermal separation (flash, distillation, or the like), for which depressurization of the highly pressurized product mixture may be necessary.

A preferred embodiment of the present invention further comprises a cooling of the product mixture before the separation in step c to a temperature between 40° C. and 100° C., preferably between 50° C. and 95° C., particularly preferably between 60° C. and 90° C. This requires a suitable cooling apparatus. The cooling is especially carried out in an output cooler. Apparatuses that have proven suitable include for example tube bundle heat exchangers, wherein the reaction mixture is preferably passed through the tubes and the cooling medium is preferably passed through the shell of the heat exchanger.

The cooling of the product mixture ensures a reduction in the catalyst metal usage factor. The problem is that during a hydroformylation and the subsequent separation a small portion of the metal, in particular of the rhodium, is always lost in various ways. Due to the high prices of the metals to be employed, in particular of the metal, this increases process costs because the losses have to be compensated by replenishment. However, the cooling according to the invention has the effect that the usage factor falls, i.e. less catalyst metal, in particular rhodium, is lost and thus less is required for replenishment. This accordingly makes it possible to considerably reduce process costs.

The removal of the homogeneous catalyst system to obtain the crude product mixture in step c may be effected with the aid of various separation processes, for example by thermal separation and/or by membrane separation. Suitable processes are familiar to those skilled in the art. It is preferable when initially a thermal separation, for example an evaporation, and subsequently a membrane separation are carried out. In the evaporation it is mainly product aldehydes (trimethylhexanal and C5 to C8 aldehyde) and unconverted diisobutenes and C4 to C7 olefins that are obtained overhead as crude product mixture. A high boiler phase containing the homogeneous catalyst, trimethylhexanal and in some cases C5 to C8 aldehydes and any high boilers formed is obtained in the bottoms. The high boiler phase may then be subjected to a membrane separation to discharge any high boilers. As is known, a membrane separation gives rise to a retentate and a permeate. The catalyst system will accumulate in the retentate. The permeate can be sent for further processing.

The retentate here contains the homogeneous catalyst system. It is preferable in accordance with the invention when the retentate is recycled to the hydroformylation in step b/to the reaction zone where the hydroformylation is carried out. This allows the catalyst system to be reused. In the preferably continuous execution of the claimed process, this gives rise to a catalyst cycle in which at most only minor process-related catalyst losses need to be compensated. If according to the preferred embodiment the diisobutene stream, the C4 to C7 olefin and the homogeneous catalyst system are mixed especially in a suitable mixing vessel prior to the hydroformylation in step b, the retentate is sent to the mixing vessel.

Any suitable membrane material may be used for the membrane separation. Preference is given to using an OSN (organic solvent nanofiltration) membrane material in the membrane separation after the evaporation of the process of the invention. Such a membrane material preferably consists at least of a separation-active layer (also: active separation layer) and a substructure on which the separation-active layer is present. The membrane material of the invention preferably consists at least of a separation-active layer and a substructure.

The substructure preferably has a porous structure that is permeable to the permeate that has passed through the separation-active layer. The substructure has a stabilizing function and serves as a support for the separation-active layer. The substructure may in principle consist of any suitable porous material. Suitable materials are familiar to those skilled in the art. A prerequisite, however, is that the material is stable to acids and bases. The substructure may consist of the same material as the separation-active layer. Preferred materials for the substructure include plastics such as polypropylene (PP), polyethylene (PE) or non-condensation polymers that are not susceptible to hydrolytic or alcoholytic cleavage, such as polysulfones, polytetrafluoroethylene (PTFE), polyethersulfone (PES), polyvinylidene fluoride (PVDF) or polyacrylonitrile (PAN).

The separation-active layer according to the invention is preferably composed of a PAEK (polyaryletherketone) polymer. PAEK has the particular feature that, within the repeat unit, aryl groups are linked alternately via an ether functionality and a ketone functionality. A separation-active layer that is preferred according to the invention is composed of PEEK (polyether ether ketone). As the separation-active layer, particular preference is given to using PEEK polymers having a degree of sulfonation of less than 20%, particularly preferably having a degree of sulfonation of less than 10%. The corresponding PEEK polymers and the production thereof are described in WO 2015/110843 A1.

The membrane separation in step c is preferably carried out at a temperature in the range from 25° C. to 100° C., further preferably in the range from 30° C. to 80° C. and particularly preferably in the range from 40° C. to 70° C. To bring the product mixture to the prevailing temperature preferred for the membrane separation, the product mixture may be cooled. In addition to active cooling using a coolant, cooling may also be achieved via a heat exchanger where heat energy is transferred to another stream, thus cooling the product mixture and heating the other stream.

The transmembrane pressure (TMP) in the membrane separation in step c is preferably in the range from 10 to 60 bar, further preferably in the range from 15 to 55 bar, particularly preferably in the range from 40 to 50 bar. The permeate-side pressure here may be above atmospheric pressure and preferably up to 15 bar, preferably 2 to 7 bar. The difference between the TMP and the permeate-side pressure gives the retentate-side pressure. In a preferred embodiment, care should be taken, in the case of the pressure ratios and the permeate-side pressure in particular, to ensure that the pressure is set such that evaporation after passage through the membrane is avoided. Evaporation could lead to unstable operation.

In the subsequent step d, the distillative processing of the crude product mixture is carried out in at least one distillation column to remove the unreacted olefins (diisobutenes and C4 to C7 olefins). This affords an aldehyde mixture comprising the aldehydes 3,5,5-trimethylhexanal and C5 to C8 aldehyde formed.

In the distillative processing of the crude product mixture in step d the unreacted olefins, i.e. unreacted diisobutenes and unreacted C4 to C7 olefins, are obtained at the top of the at least one distillation column. The mixture of the aldehydes formed thus accumulates in the bottom of the at least one distillation column. The tops stream comprising the unreacted olefins may be recycled to the hydroformylation in step b/to the reaction zone. If the components used undergo mixing prior to the hydroformylation, the overhead stream is by definition supplied to the mixing. This allows continuous operation of the process of the invention at the highest possible yield. A purge can be withdrawn from the recycled overhead stream in order to discharge low-boiling by-products from the process.

The distillative processing to remove the unreacted olefins in step d may be carried out in a distillation column. It would be conceivable for the distillative processing to remove the unreacted olefins in step d to be carried out in two or more distillation columns. However, this would entail markedly higher apparatus costs. It is therefore preferable that the distillative processing in step d is carried out in a single distillation column.

The pressure in the distillation column in the distillative processing in step d is preferably in the range from 0.3 to 2 bar, more preferably in the range from 0.4 to 1 bar, particularly preferably in the range 0.5 to 0.7 bar. The temperature in the bottom of the distillation column in the distillative processing in step d is preferably in the range from 80° C. to 160° C. The temperature at the top of the distillation column in the distillative processing in step d is preferably in the range from 30° C. to 80° C. In addition, it is preferable when the reflux ratio in the distillation column is between 1 and 2. The distillation column for the removal in step d preferably comprises 10 to 30 theoretical plates. The distillation column may contain high-performance structured packings. Suitable high-performance structured packings are known to those skilled in the art.

As mentioned above the distillative processing affords an aldehyde mixture comprising the aldehydes formed from the diisobutene and the C4 to C7 olefin. To obtain both aldehydes in the highest possible purity a further distillation step may be performed to separate the aldehydes from one another. The aldehyde from the C4 to C7 olefin will be obtained at the top of the distillation column and the aldehyde from the diisobutene at the bottom of the distillation column. Also conceivable are dividing wall columns in order at various points to obtain the aldehydes as pure components within a column. Appropriate technical configurations of a dividing wall column are familiar to those skilled in the art and available on a large industrial scale.

The present process is particularly suitable for hydroformylation of diisobutene and C4 to C7 olefins. Certain combinations of olefins are particularly preferred in the context of the present invention:

In a preferred embodiment, the process relates to the hydroformylation of diisobutene and a C4 olefin, i.e. 1-butene, cis- and/or trans-2-butene, isobutene or mixtures thereof. The aldehyde formed from the diisobutene is 3,5,5-trimethylhexanal. Pentanal, 2-methylbutanal or 3-methylbutanal is formed from the C4 olefin. Employing a mixture of butenes accordingly also affords a mixture of the recited aldehydes.

In a particularly preferred embodiment, the process relates to the hydroformylation of diisobutene and isobutene. The aldehyde formed from the diisobutene is 3,5,5-trimethylhexanal. 3-Methylbutanal is formed from the isobutene.

The present invention is explained hereinbelow by reference to examples. The examples relate to preferred embodiments but are not to be understood as limiting the invention.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The entire disclosures of all applications, patents and publications, cited herein and of corresponding European application No. 23186740.9, filed Jul. 20, 2023, are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. Process for hydroformylation of diisobutene and a C4 to C7 olefin, wherein the process comprises at least the following steps:
a. providing a diisobutene stream containing 2,4,4-trimethylpent-2-ene and 2,4,4-trimethylpent-1-ene and providing an olefin stream containing the C4 to C7 olefin;
b. hydroformylation of diisobutene and the C4 to C7 olefin with synthesis gas in the presence of a homogeneous catalyst system comprising at least Co or Rh and optionally a phosphorus-containing ligand in a reaction zone to obtain a preferably liquid product mixture comprising at least the aldehydes 3,5,5-trimethylhexanal and a C5 to C8 aldehyde formed by the hydroformylation, the homogeneous catalyst system and unreacted olefins;
c. removing the homogeneous catalyst system from the preferably liquid product mixture to obtain a crude product mixture comprising at least the aldehydes 3,5,5-trimethylhexanal and a C5 to C8 aldehyde formed by the hydroformylation and the unreacted olefins; and
d. distillative processing of the crude product mixture in at least one distillation column to remove the unreacted olefins to obtain an aldehyde mixture containing the aldehydes 3,5,5-trimethylhexanal and a C5 to C8 aldehyde formed.

2. Process according to claim 1, wherein the hydroformylation in step b is performed at a temperature of 90° C. to 250° C., preferably of 120° C. to 200° C., particularly preferably of 120° C. to 170° C.

3. Process according to claim 1, wherein the hydroformylation in step b is performed at the pressure of 100 to 350 bar, preferably 175 to 325 bar, particularly preferably 200 to 300 bar.

4. Process according to claim 1, wherein the proportion of 2,4,4-trimethylpent-1-ene in the diisobutene stream is at least 60 mol %, preferably at least 70 mol %.

5. Process according to claim 1, wherein the removal of the homogeneous catalyst system in step c is effected by thermal separation and/or membrane separation.

6. Process according to claim 1, wherein the removal of the homogeneous catalyst system in step c is effected by evaporation and subsequent membrane separation.

7. Process according to claim 1, wherein the removal of the homogeneous catalyst system in step c is effected by membrane separation.

8. Process according to claim 7, wherein the homogeneous catalyst system accumulates in the retentate.

9. Process according to claim 1, wherein the diisobutene stream, the C4 to C7 olefin stream and the homogeneous catalyst system are initially mixed in a mixing vessel before they are passed into the reaction zone.

10. Process according to claim 1, wherein the distillative processing in step d is carried out in a single distillation column.

11. Process according to claim 10, wherein the pressure in the distillation column is in the range from 0.3 to 2 bar, preferably in the range from 0.4 to 1 bar, particularly preferably in the range from 0.5 to 0.7 bar.

12. Process according to claim 10, wherein the temperature in the bottom of the distillation column is in a range from 80° C. to 160° C.

13. Process according to claim 10, wherein the temperature at the top of the distillation column is in a range from 30° C. to 80° C.

14. Process according to claim 1, wherein in step d the unreacted olefins are removed and recycled to hydroformylation step b.

15. Process according to claim 1, wherein a C4 olefin, preferably isobutene, is employed, as a result of which the aldehyde mixture obtained is a mixture of 3,5,5-trimethylhexanal and pentanal, 2-methylbutanal or 3-methylbutanal, preferably 3-methylbutanal.
